(19) United States
(12) Patent Application Publication
(10) Pub. No.: US 2004/0115236 A1
(43) Pub. Date: Jun. 17, 2004

(54) Devices and methods for management of inflammation

(76) Inventors: Tai Wah Chan, Palo Alto, CA (US); Randolph M. Johnson, Half Moon Bay, CA (US); Andrew R. Miksztal, Palo Alto, CA (US); Arthur J. Tipton, Birmingham, CA (US); John W. Gibson, Springville, AL (US); Stacey K. Meador, Vance, AL (US)

Correspondence Address: DURECT CORPORATION, 10240 BUBB ROAD, CUPERTINO, CA, 95014, US

(21) Appl. No.: 10/433,131

(22) PCT Filed: Oct. 9, 2001
(86) PCT No.: PCT/US01/31652

(30) Foreign Application Priority Data
Oct. 6, 2000 (US) 60238575

(51) Int. Cl. A61K 31/353; A61K 31/7012; A61K 9/22; A61F 2/00

(52) U.S. Cl. 424/423; 514/53; 604/890.1; 514/456

(57) ABSTRACT

Devices and methods for the sustained release into a subject of an inhibitor of mast cell-mediated inflammation, specifically sustained-release of sodium cromoglycate as a prophylactic to prevent acute asthma attacks.

### FIELD OF THE INVENTION

The invention relates to devices and methods designed to facilitate sustained-release of a drug that modulates an inflammatory response.

### BACKGROUND OF THE INVENTION

Inflammation And Its Treatment

The inflammatory response in a host is an important biological reaction for interruption and resolution of an infectious process. Inflammation is the localized response of vascularized tissues to injury caused by chemical, physical or biological agents. Clinically, the signs of inflammation include redness, swelling, heat, pain, loss of function and fever. Chronic inflammation in any tissue will eventually lead to the destruction of the tissue and may also lead to clinical disease associated with this tissue destruction. Many disorders are associated with inappropriate inflammation, including asthma, autoimmune disorders (e.g., arthritis and lupus erythematosus), allergic ocular disorders (e.g., conjunctivitis), mastocytosis, inflammatory bowel disease and Crohn's disease.

Asthma is caused by chronic inflammation of bronchial airways. Approximately 14.6 million Americans currently suffer from asthma, and approximately 4.8 million of those affected are children under age 18. Vital and Health Statistics, (1995) 10:193. The prevalence of asthma is on the rise, with approximately 5.4 percent of Americans reported to suffer from asthma in 1994, a 75 percent increase since 1980. Surveillance for asthma—United States 1960-1995, Morbidity and Mortality Weekly Report, (1998); 47:SS-1. The rate of asthma is rising especially quickly in young children, with 5.8 percent of children under age 5 suffering from asthma in 1994 (as reported by a family member), a 160 percent increase since 1980. Currently, asthma is responsible for more than 500 deaths and 466,000 hospitalizations each year. Asthma-related health care costs are estimated at more than $6 billion a year. “HHS Targets Efforts on Asthma,” Department of Health and Human Services, May 21, 1998.

Mast cells play a key component of many types of inflammation, including asthma. When mast cells are activated, they release many chemical messengers. These chemical messengers include cytokines, biogenic amines, such as histamines, and lipid mediators, such as platelet activating factor (PAF), prostaglandin D₂ (PGD₂) and leukotriene C (LTC). These chemicals activate vascular leakage of cells and fluids, broncho-constriction, intestinal hypermotility, inflammation, and tissue remodeling.

Biogenic amines (also called vasoactive amines) are non-lipid low molecular weight compounds that possess an amine group. In human mast cells, the only mediator of this class present in significant quantities is histamine, though in rats, serotonin is present in considerable amounts. Histamine binds to target cell receptors and initiates intracellular events such as phosphatidylinositol breakdown to IP₃ and DAG. Binding of histamine to endothelium leads to cell contraction and leakage of plasma into surrounding tissues. Histamine also stimulates endothelial cells to make smooth muscle relaxants such as PGI₂ and nitric oxide, causing well-characterized vasodilation effects (Cellular and Molecular Immunology 4^{th} ed., Abbas et al., Saunders, pub).

[image]

A number of asthma therapies are currently used, each attempting to target various mechanisms to combat this chronic inflammatory disease of the airways. Currently, the combination of short-term and long-term (acute and chronic) medications is important for the successful management of asthma. Long-term control medications include corticosteroids, inhibitors of mast cell activation/degranulation (e.g., sodium cromoglycate or nedocromil sodium), long-acting beta-2-agonists, methylxanthines, and leukotriene modifiers. Quick-relief medications include short-acting beta2-agonists, anticholinergics, and systemic corticosteroids.

Mast cells and mast cell activation/degranulation are vital to the development of allergic inflammatory reactions. Compounds with the ability to stabilize mast cells, prevent mast cell activation/degranulation, and/or affect the cascade of inflammation-promoting events arising from mast cell activation (e.g., recruitment of immune cells such as eosinophils) are attractive therapeutics. For example, inhibitors of mast cell-mediated inflammation do not have the systemic side effects of more broadly acting immunosuppressants (e.g., corticosteroids), such as suppression of the hypothalamic-pituitary-adrenal (HPA) axis, adverse effects on bone metabolism (e.g., osteoporosis), slowing of growth in children and adolescents, bruising, psychological changes, ocular hypertension, wide-angle glaucoma and cataract formation. Thus, inhibitors of mast cell-mediated inflammation (e.g., sodium cromoglycate or nedocromil sodium) provide an alternative to corticosteroid therapy. In addition, inhibitors of mast cell-mediated inflammation may be used as a conjunctive therapy with lowered dosage of corticosteroids, especially in patients where there is concern for toxicity of inhaled corticosteroids.

Sodium cromoglycate is one of the more commonly used prophylactic drugs and is thoroughly described at page 667 of The Pharmacological Basis of Therapeutics by Goodman and Gilman, 9^{th} ed., McGraw Hill. Sodium cromoglycate (or, in the United States “cromolyn sodium” or “cromolyn”) is a chromone complex, possessing the typical two-ring chromone structure (see Definitions section). Sodium cromoglycate acts by inhibiting the release of chemical mediators (e.g., cytokines, biogenic amines, such as histamines, and lipid mediators, such as PAF, PGD₂, LTC) from sensitized mast cells. Sodium cromoglycate reduces inflammation sneezing, nasal discharge, nasal congestion, and eye irritation.

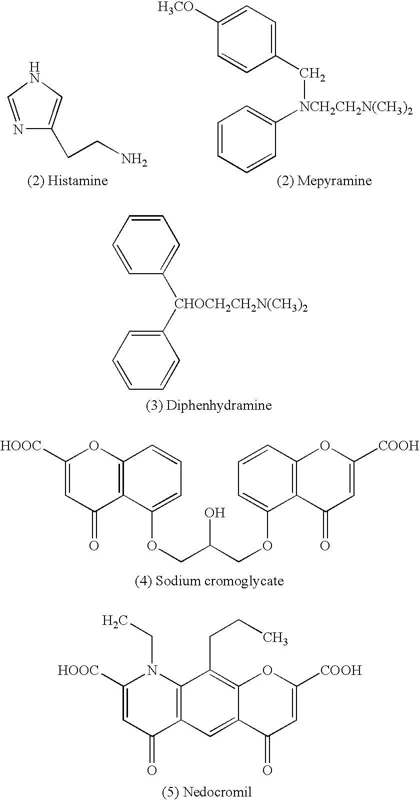

When administered prior to allergen exposure, sodium cromoglycate blocks both the early asthmatic response (EAR) and late asthmatic response (LAR). Sodium cromoglycate does not exhibit smooth muscle relaxant properties and, therefore, is not a bronchodilator; it has long been held that the primary mechanism of action appears to be inhibition of mediator release from mast cells.

Although the exact mechanism by which sodium cromoglycate inhibits mast cell mediator release is not known, it is thought to inhibit calcium influx into mast cells by phosphorylation of a membrane protein.

Sodium cromoglycate has been effective in clinical studies and in pediatric asthma, allergic asthma and exercise-induced asthma (EIA). A number of studies have evaluated the effect of sodium cromoglycate on nonspecific bronchial hyperreactivity. Inhaled administration of 20 mg doses of sodium cromoglycate via Spinhaler yields peak plasma concentrations of 35-90 ng/ml. With inhaled administration, plasma concentrations of 4-6 ng/ml yield 66% inhibition of exercise-induced bronchoconstriction; concentrations larger than 4-6 ng/ml yield no additional benefit for the inhibition of exercise-induced bronchoconstriction. Administration of sodium cromoglycate for longer than 6 weeks has been shown to result in an improvement in airway reactivity. A 3 month, double-blind, placebo-controlled clinical trial found that sodium cromoglycate is effective for chronic adult asthma (Petty T. L. et al, Am. Rev. Respir. Dis. (1989) 139:694-701), and other studies have demonstrated effective symptom control with sodium cromoglycate in children (Shapiro G. G. et al., J. Allergy Clin. Immunol. (1991) 88:742-8; Maykoski-Bell and Quincy, Minn. J. Asth. Res. (1972) 3131:42, Alh&Jlh, pub). It is estimated that about 60% to 79% of asthmatics will respond favorably to sodium cromoglycate therapy.

Nedocromil sodium is a pyranoquinolone derivative that inhibits the release of inflammatory mediators from mast cells, inhibits recruitment of other inflammatory cells (such as eosinophils and neutrophils) into the airway epithelium, inhibits both immediate and late bronchoconstriction, and decreases bronchial hyperresponsiveness.

A number of studies suggest that nedocromil sodium blocks both the BAR and LAR to a variety of allergic and nonallergic asthmatic triggers. While the efficacies of nedocromil sodium and sodium cromoglycate are similar in situations involving allergen exposure, nedocromil sodium is generally more potent than sodium cromoglycate in protecting against nonallergic exposures. Nedocromil sodium has been shown to acutely inhibit the bronchospasm induced by several kinds of challenges, including exercise. Sodium cromoglycate is effective for prophylaxis of asthma but not for treatment of acute exacerbation, i.e., once an attack has started, it is too late.

Inhalation is by far the most common route of administration for the prophylaxis of asthma. Many patients find that inhalation of sodium cromoglycate is irritating and commonly leads to a severe sneezing attack This has limited patients' acceptance of cromoglycate as a popular therapy. Sodium cromoglycate is also available in a oral form for the treatment of mastocytosis, as a nasal spray for the treatment of seasonal rhinitis, and in topical form for the treatment of kerato-conjunctivitis.

Although inhalation has been by far the most common route of administration, additionally, for pharmacokinetic research purposes only, sodium cromoglycate has been administered intravenously. (Fuller R W, Collier J G: The Pharmacokinetic Assessment Of Sodium Cromoglycate, J. Pharm. Pharmacol. 1983; 35: 289-92; and Neale M G, et al: The Pharmacokinetics Of Sodium Cromoglycate In Man After Intravenous And Inhalation Administration, Br. J. Clin. Pharmacol. 1986; 22: 373-82.)

The Fuller study discloses intravenous delivery of sodium cromoglycate via a vein in the forearm over a period of one minute, after which samples were drawn over a two hour period.

The Neale study discloses slow intravenous delivery of sodium cromoglycate via a vein in the forearm over a period of thirty minutes.

The safety profile of sodium cromoglycate is very favorable (please see the Internet Drug Index, www.rxlist.com/cgi/generic2/cromolyn.htm). Clinical experience with the use of sodium cromoglycate suggests that adverse reactions are rare events. The following adverse reactions have been associated with inhalation of sodium cromoglycate spray solution: cough, nasal congestion, nausea, sneezing, and wheezing. Other reactions (drowsiness, nasal itching, nosebleed, nose burning, serum sickness, and stomachache) have been reported in clinical trials, but a causal relationship could not be established. In addition, adverse reactions have been reported with inhalation of sodium cromoglycate powder. The most common side effects are associated with inhalation of the powder and include transient cough (1 in 5 patients) and mild wheezing (1 in 25 patients). These effects rarely require treatment or discontinuation of the drug.

Current treatment of asthma includes many medications including beta agonists, steroids, and leukotriene inhibitors. Although these medications are effective for many patients, several factors may limit their efficacy. First, side effects are common, particularly with steroids, and may limit dosing. Second, patients often fail to take their medications.

Compounds such as sodium cromoglycate and nedocromil sodium are poorly absorbed orally with an oral bioavailability of less than 1%. Conventional therapeutics are only provided by inhalants, which, likewise, have low bioavailability (about 7.5%).

Therapeutic regimes using these agents often require as many as 4-6 inhaled doses per day. The required frequency of dosing makes compliance with scheduled dosages difficult, especially with children and the elderly population. Even where patient compliance is perfect, use of inhalers does not provide for a continuous dose of the drug, but rather delivers drug in a bolus. This results in the drug being present in a subject in amounts greater than needed for a therapeutic effect (e.g., at the time of initial delivery of a bolus) and in amounts well under the therapeutic threshold (e.g., at times between dosages).

Inhaled agents also do not work well in people who have nasal polyps or other defects that could block the spray from reaching the lining of the nose and sinuses, or for people with severely restricted air-flow through the nasal passages such as people with severely swollen turbinate tissue, for example caused by an allergy. In those suffering from chronic respiratory inflammation, congestion and bronchoconstriction can reduce the amount of the therapeutic that can be effectively administered to a subject. In addition, administration is dependent upon proper use of the inhalation devices, such as metered dose inhalers, and incorrect use of such devices adversely affects proper dosing.

Although both sodium cromoglycate and nedocromil sodium are useful in the prophylactic treatment of both allergic and exercise-induced asthma, neither can stop an established asthmatic attack. In fact, once therapy with these compounds is started, it may take 1 to 4 weeks before a full effect is felt. Since effectiveness of the drug requires administration before symptoms are present, compliance is extremely important but difficult to achieve.

Inhalation therapy of sodium cromoglycate or nedocromil sodium is often associated with irritation of the throat and trachea, further reducing compliance among those not currently exhibiting asthma-associated symptoms. Moreover, the limited bioavailability through oral routes limits the use of oral dosage forms of these compounds in the treatment of mast cell mediated inflammatory disorders. Sodium cromoglycate and nedocromil sodium are thus not currently used in the treatment of a wide variety of mast cell mediated inflammatory responses.

Drug Delivery Devices

Drug delivery devices are generally split into five major groups: inhaled, oral, transdermal, parenteral and suppository. Inhaled devices include gaseous, misting, emulsifying and nebulizing bronchial (including nasal) inhalers; oral devices include mostly pills; whereas transdermal devices include mostly patches. Parenteral devices are split into two sub-groups: injectable and non-injectable devices. Non-injectable devices are generally referred to as “implants” and include pumps and solid biodegradable and non-biodegradable polymers. Injectable devices are split into bolus injections, that are injected and dissipate, releasing a drug all at once, and depots, that remain discrete at the site of injection, releasing drug over time. Depots include e.g., oils, gels, liquid polymers and non-polymers, and microspheres.

There has been extensive research in the area of biodegradable controlled release systems for bioactive compounds. Biodegradable matrices for drug delivery are useful because they obviate the need to remove the drug-depleted device. The most common matrix materials for drug delivery are polymers. The field of biodegradable polymers has developed rapidly since the synthesis and biodegradability of polylactic acid was reported by Kulkami et al., in 1966 (Polylactic Acid For Surgical Implants, Arch. Surg., 93:839). Examples of other polymers which have been reported as useful as a matrix material for delivery devices include polyanhydrides, polyesters such as polyglycolides and polylactide-co-glycolides, polyamino acids such as polylysine, polymers and copolymers of polyethylene oxide, acrylic terminated polyethylene oxide, polyamides, polyurethanes, polyorthoesters, polyacrylonitriles, and polyphosphazenes. See, for example, U.S. Pat. Nos. 4,891,225 and 4,906,474 to Langer (polyanhydrides), U.S. Pat. No. 4,767,628 to Hutchinson (polylactide, polylactide-co-glycolide acid), and U.S. Pat. No. 4,530,840 to Tice, et al. (polylactide, polyglycolide, and copolymers).

Degradable materials of biological origin are well known, for example, crosslinked gelatin. Hyaluronic acid has been crosslinked and used as a degradable swelling polymer for biomedical applications (U.S. Pat. No. 4,957,744; Surface Modification Of Polymeric Biomaterials For Reduced Thrombogenicity, Polym. Mater. Sci. Eng., 62:731-735).

Biodegradable hydrogels have also been developed for use in controlled drug delivery as carriers of biologically active materials such as hormones, enzymes, antibiotics, antineoplastic agents, and cell suspensions. Temporary preservation of functional properties of a carried species, as well as the controlled release of the species into local tissues or systemic circulation, have been achieved. See for example, U.S. Pat. No. 5,149,543. Proper choice of hydrogel macromers can produce membranes with a range of permeability, pore sizes and degradation rates suitable for a variety of applications in surgery, medical diagnosis and treatment.

Many dispersion systems are currently in use as, or being explored for use as, carriers of substances, particularly biologically active compounds. Dispersion systems used for pharmaceutical and cosmetic formulations can be categorized as either suspensions or emulsions. Suspensions are defined as solid particles ranging in size from a few nanometers up to hundreds of microns, dispersed in a liquid medium using suspending agents. Solid particles include microspheres, microcapsules, and nanospheres. Emulsions are defined as dispersions of one liquid in another, stabilized by an interfacial film of emulsifiers such as surfactants and lipids. Emulsion formulations include water in oil and oil in water emulsions, multiple emulsions, microemulsions, microdroplets, and liposomes. Microdroplets are unilamellar phospholipid vesicles that consist of a spherical lipid layer with an oil phase inside, as defined in U.S. Pat. Nos. 4,622,219 and 4,725,442. Liposomes are phospholipid vesicles prepared by mixing water-insoluble polar lipids with an aqueous solution. The unfavorable entropy caused by mixing the insoluble lipid in the water produces a highly ordered assembly of concentric closed membranes of phospholipid with entrapped aqueous solution.

U.S. Pat. No. 4,938,763, discloses a method for forming an implant in situ by dissolving a non-reactive, water insoluble thermoplastic polymer in a biocompatible, water soluble solvent to form a liquid, placing the liquid within the body, and allowing the solvent to dissipate to produce a solid implant. The polymer solution can be placed in the body via syringe. The implant can assume the shape of its surrounding cavity. In an alternative embodiment, the implant is formed from reactive, liquid oligomeric polymers which contain no solvent and which cure in place to form solids, usually with the addition of a curing catalyst.

The term “microspheres” (also known as “microparticles” or nanospheres” or “nanoparticles”) refers to small particles, typically prepared from a polymeric material and typically no greater in size than about 10 micrometers in diameter. For reference, please refer generally to Encyclopedia of Controlled Drug Delivery 1999, published by John Wiley & Sons Inc, edited by Edith Mathiowitz. For example, U.S. Pat. No. 4,994,281 discloses polylactic acid microspheres, prepared by the in-water drying method, containing a physiologically active substance and having an average particle size of about 0.1 to 10 micrometers.

Microparticles can be prepared by grinding to the appropriate particle size a mixture of biodegradable polymer and drug. The mixture may be prepared by a melt or solvent blend. Microspheres may be prepared by a number of methods familiar to those skilled in the art including spray drying, coacervation and emulsion techniques. For example the methods described in U.S. Pat. No. 6,291,013 where a polymer solution containing drug is emulsified in water and then the solvent is removed by extraction, evaporation or a combination of the two may be used.

Pumps, of course, are well known in the art, and include external and implanted pumps that generally include a closed reservoir for containment of drug, and some pumping mechanism use to generate pressure that forces the drug from the reservoir, through an orifice, into subject, either directly or via a catheter. Implanted pumps include osmotic pumps, vapor pressure pumps, electrolytic pumps, effervescent pumps, piezoelectric pumps, electrochemical pumps and others. An exemplary osmotic pump is the DUROS® pump developed by Alza Corp of Palo Alto, Calif. Another system of particular interest is the “elementary osmotic pump” that is described in a number of issued U.S. patents, for example U.S. Pat. Nos. 3,845,770 and 3,916,899 and other patents related thereto.

Present Need For New Treatments

If the disadvantages associated with inhaled dosing of inhibitors of mast cell mediated inflammation, such as sodium cromoglycate, could be eliminated, and bioavailability and dosing compliance could be improved, such drugs might become more popular for the prophylaxis of asthma. Were this to occur, it is possible that patients might benefit in two ways. First, patients who require steroids might be able to decrease their intake of these compounds, thereby decreasing the occurrence of steroid-induced side effects. Second, if asthma drugs such as sodium cromoglycate could be administered in an extended-release form, e.g., as a subcutaneous depot, issues related to compliance could be traversed, thereby improving patient well being and improving drug marketability.

As is evident from the above, there is a great need for devices and methods for effective and practical long-term management of inflammation, and in particular with mast cell-mediated inflammation in conditions such as asthma, autoimmune disorders, allergic ocular disorders, mastocytosis, inflammatory bowel disease, and Crohn's disease.

There is a particular need for devices and methods for the long-term management of respiratory inflammation, such as that associated with chronic or recurring asthma, that provide better efficacy, patient compliance and reduced side effects. In particular there is a need for an extended-release dosage form whereby sodium cromoglycate or other immune modulator is released over a period of weeks or months. The present invention addresses these needs.

### SUMMARY OF THE INVENTION

The invention features devices and methods for the delivery of an inhibitor of mast cell-mediated inflammation such as a chromone (e.g., sodium cromoglycate) using a sustained-release dosage form. A sustained-release dosage form comprises a drag and a drug delivery device. The drug delivery device may be a depot or non-injectable implant that can be injected or otherwise implanted in a subject.

The dosage form may be placed at or near to the site of intended action (the target site) e.g., the lung or joint, or alternatively, the dosage form may be placed at a site in the body that is distant from the intended site of action, e.g., subcutaneously, for example in the upper arm.

A drug delivery device of the invention may be based on, for example, diffusive, erodible or convective systems, e.g., pumps, (such as osmotic pumps, vapor pressure pumps, electrolytic pumps, effervescent pumps, piezoelectric pumps, or electrochemical pumps), that may or may not be connected to a catheter, and also biodegradable non-injectable implants, electrodiffusive systems, electroosmotic systems, erosion-based systems, electromechanical systems, injectable liposomes, microspheres or ant depot.

In certain embodiments a catheter can be used in conjunction with the dosage form that allows the drug to be transported from the site of implantation or injection to the intended site of action (e.g., lung tissue).

Alternatively, the drug can reach the intended site of action in the body via the systemic circulation, i.e., the drug is released from the sustained-release dosage form, diffuses through tissue into adjoining circulatory vessels, and enters the systemic circulation which transports the drug to the site of action in the body to modulate the inflammatory response (e.g., inflammation of the lung, sinuses, bowel, or other site).

Drug release rates for the invention are generally low, from about 0.01 microliters/day to 2 ml/day, or about 0.01 micrograms per day up to about 20 milligrams per day. In one embodiment, a drug delivery device provides for substantially continuous, subcutaneous delivery of drug at a rate of from about 0.1 μg/hr to about 200 μg/hr and in some instances up to about 833 μg per hour, usually from about 0.25 μg/hr or 3 μg/hr to about 85 μg/hr, and typically between about 5 μg/hr to about 100 μg/hr. In a specific exemplary embodiment, sodium cromoglycate is delivered at a rate of from about 0.1 μg/hr, 0.25 μg/hr, 1 μg/hr, generally up to about 833 μg/hr. Appropriate amounts of a particular inhibitor of mast cell-mediated inflammation can be readily determined by the ordinarily skilled artisan based upon, for example, the relative potency of these drugs. The actual dosage depends on a number of factors such as potency, bioavailability, and toxicity. Low volume rate drug delivery avoids accumulation of drug at the target site (e.g., pooling effect) by providing for a rate of administration that is less than, the same as, or only very slightly greater than the rate of removal of drug from the target site (e.g., by absorption of drug in tissues at the site, movement of drug away from the site by flow of blood or other bodily fluids, etc.). Thus, in addition to providing an implantable system for delivery of drugs such as inhibitors of mast cell-mediated inflammation (e.g., sodium cromoglycate or nedocromil sodium), the present invention also provides a method for treating inflammation by elegantly balancing the rates of drug absorption and drug delivery to accomplish administration of a therapeutically effective amount of drug, while avoiding accumulation of drug at the target site.

The drug delivery device can be any device implantable by injection or by other means. Non-injectable implants may include: an osmotic pump, an electrochemical pump, an electromechanical pump, an electroosmotic pump, a piezoelectric pump, an effervescent pump, a vapor pressure pump, an electrolytic pump, a hydrolytic system, an electrodiffusion system, an elastomeric system, an osmotic bursting matrix, or a biodegradable implant. Injectables may include: a sustained release injectable, a microparticulate suspension, a liposome formulation, a SAIB depot, an oil suspension, an encapsulated particulate suspension system, and microsphere system, a biodegradable implant, an erosion-based system, or any sort of depot. Depots may include, for example, injectable non-polymeric materials such as those described in U.S. Pat. Nos. 6,051,558; 5,747,058; and 5,968,542, such as a high viscosity liquid, such as a non-polymeric non-water-soluble liquid carrier material, e.g., Sucrose Acetate Isobutyrate (SAIB) or another compound such as a compound described in U.S. Pat. No. 5,747,058.

Administration of a formulation by parenteral delivery according to the invention is particularly preferred where delivery by inhalation is undesirable, e.g., the subject has difficulty with compliance with the desired dosage regime. In particular, sustained release dosage forms are convenient for long-term drug administration and can allow drug therapy to be conducted on an outpatient basis where the patient's health allows such. Long-term parenteral delivery also increases patient compliance. Implantable dosage forms, e.g., osmotic pumps and depots, have an added benefit in that they reduce the risk of infection associated with external pumps or other methods that require repeated breaking of the skin and/or maintenance of a port for administration.

The invention provides two broad major advantages over current therapies. First, patients who require steroids will be able to decrease their intake of these compounds, thereby decreasing the occurrence of steroid-induced side effects. Second, because sodium cromoglycate is administered in an extended-release form, e.g., as a subcutaneous depot, issues related to compliance could be bypassed, thereby improving patient well-being.

One significant specific advantage of the invention is that the invention can be used to deliver relatively small quantities of inhibitors of mast cell-mediated inflammation accurately and precisely over a selected period of time. Use of a long-term drug delivery device obviates the need for regular dosing by the patient, thus increasing patient compliance with a prescribed therapeutic regimen, and in particular compliance with a prophylactic regimen prescribed prior to the onset of symptoms. It is well established that compliance is a severe problem for prophylactic treatments in which patients are required to take medicines while asymptomatic for protracted or indefinite periods. This is especially useful in populations in which compliance with such medications can be more difficult, for example with elderly patients and with children, mentally subnormal patients, or patients who are senile, demented, uneducated, itinerant, morally delinquent or uncooperative. Long-term delivery from an implanted dosage form provides an effective and inexpensive method of providing prophylactic care to such populations.

Another notable advantage of the invention is that parenteral administration avoids the need for inhalation of the therapeutic agent, which is known to be very irritating in some cases, and can be difficult in a subject suffering from bronchoconstriction or nasal obstructions. Parenteral administration can increase the efficacy of the drug as compared to inhaled therapies, as the intended dosage may be maintained at the target site.

Another notable advantage is that parenteral delivery of inhibitors of mast cell-mediated inflammation provides almost complete bioavailability. This is a significant advantage, as known inhibitors of mast cell-mediated inflammation (e.g., mast cell stabilizers sodium cromoglycate and nedocromil sodium) have very poor bioavailability orally, and low bioavailability via inhaled and ocular routes, with conventional treatments requiring multiple doses of drug per day to provide adequate bioavailability.

Another advantage of the invention is that an inhibitor of mast cell-mediated inflammation can be delivered into the systemic circulation in a patterned fashion, e.g., continuously with accuracy and precision and at low quantities as to permit long-term use of such compounds to treat and/or prevent inflammation. Continuous, low-level delivery of anti-inflammatory drugs, such as sodium cromoglycate is particularly useful because inflammation, such as that present during an asthmatic attack, is thought to result from a cascade, triggered when mast cell receptor dimerization reaches a certain threshold density. Below the triggering level, the cascade is not activated, but when the triggering level is reached, a cascade of signal amplification occurs, leading to mast cell activation/degranulation and massive histamine release. Thus one may well be disposed to reason that constant, low-level systemic release of sodium cromoglycate would be an effective way of preventing the triggering threshold from being reached, therefore preventing asthmatic attacks.

Another advantage of the invention is that sustained-release delivery of small quantities of an inhibitor of mast cell-mediated inflammation is effective in long-term prophylaxis of asthma.

Yet another advantage is that the invention provides for precise delivery of the selected inhibitor of mast cell-mediated inflammation at consistent delivery volume rates (e.g., on the order of microliters to milliliters per hour). The dosage is not dependent on a subject's correct use of the device, and so is not subject to the fluctuations is dosage that can be found with inhalation.

Still another advantage is that the invention may decrease the severity or incidence of side effects normally associated with use of other anti-inflammatories such as corticosteroids.

A further advantage is that a depot dosage form is cheap to manufacture and easy to administer, and once administered, is unobtrusive and causes no discomfort to the patient.

These and other objects, advantages and features of the present invention will become apparent to those persons skilled in the art upon reading the details of the methodology and compositions as more fully set forth below.

### BRIEF DESCRIPTION OF THE DRAWINGS

FIG. 1 illustrates systemic delivery of a drug using an implanted drug delivery device implanted subcutaneously in the upper arm of a subject.

FIG. 2 is a cut-away view of a drug delivery device (a pump).

FIG. 3 is a cut-away view of a pump with a catheter.

FIG. 4 is a graph showing cumulative release of sodium cromoglycate from a SAIB depot formulation (50/50 SAIB/benzyl benzoate with 5% by weight sodium cromoglycate).

FIG. 5 is a graph showing cumulative release of sodium cromoglycate from a monolithic polymer implant.

FIG. 6 is a graph showing the effect of sheath thickness Ro/Ri) on the release of cromolyn from coaxial implants (45% by weight sodium cromoglycate/PCL Core, with 30% by weight sodium cromoglycate/PCL Membrane).

### DESCRIPTION OF THE PREFERRED EMBODIMENTS

Before the present invention is described, it is to be understood that it is not limited to the specific methodology, devices, formulations, or conditions described, and that the terminology is not intended to limit the scope of the present invention, which will be limited only by the claims.

Also, the singular forms “a”, “and”, and “the” include their plurals, unless the context clearly dictates otherwise. Thus, reference to “a drug delivery device” includes a plurality of such devices and reference to “the method of delivery” includes reference to equivalent steps and methods known to those skilled in the art, and so forth.

Unless defined otherwise, all technical and scientific terms used herein have their usual meaning in the art to which the invention belongs. All publications mentioned are incorporated herein by reference for the purpose of describing things relevant to their context.

Definitions And Abbreviations

PFT stands for pulmonary function testing.

FEV stands for forced expiratory flow volume.

SAIB stands for sucrose acetate isobutyrate.

The term “inflammation” as used herein refers to inflammation of any tissue in the body, e.g., respiratory inflammation refers to inflammation of the respiratory system, including any pulmonary inflammation (e.g., bronchial inflammation), inflammation of nasal passages, tracheal inflammation, and the like.

The phrase “method for inhibiting inflammation” refers to a method/methods for preventing inflammation from happening, or for reducing the amount of inflammation that would otherwise occur under certain conditions, or for reducing inflammation that is already in progress in a subject.

The term “subject” is meant to encompass any subject, generally a mammal (e.g., human, canine, feline, equine, bovine, ovine, porcine, ursine, ungulate, etc).

The term “asthma” is meant a condition generally characterized by denudation of airway epithelium, deposition of collagen beneath the basement membrane, edema, mast cell activation, and infiltration by inflammatory cells, including neutrophils, eosinophils, and lymphocytes. In some patients, persistent pulmonary function abnormalities develop as a result of fibrotic changes in the basement and subbasement membranes of the airways. The inflammation contributes to bronchial hyperreactivity, limitation of airflow, and all the symptoms and signs characteristic of the disease, including wheezing, breathlessness, chest tightness, and coughing. Asthma also can be associated with acute bronchoconstriction, airway edema, mucus plug formation, and airway remodeling that lead inexorably to bronchial obstruction.

The term “chromone” refers to substances with the following core structure:

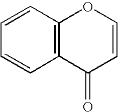

These substances modulate the immune system and include sodium cromoglycate, disodium cromoglycate and pyranoquinolones such as nedocromil sodium. Other chromones include:

| | |
|---|---|
| 3-Bromo-6-Chlorochromone | 4-Chromanone |
| 3-Bromo-6-Chloro-7-methylchromone | Chromone |
| 3-Bromochromone | Chromone-2-carboxylic acid |
| 6-Bromochromone | Chromone-3-carboxylic acid |
| 6-Bromochromone-2-carboxylic acid | 3-Cyanochromone |
| 3-Bromo-6,8-dichlorochromone | 3-Cyano-6-methylchromone |
| 6-Bromo-3-formylchromone | 6,8-Dibromochromone |
| 6-Chlorochromanone | 6,8-Dibromo-3-formylchromone |
| 6-Chlorochromone | 6,8-Dichlorochromone |
| 6-Chloro-3-cyano-7-methylchromone | 6,8-Dichloro-3-formylchromone |
| 6-Chloro-3-formylchromone | 3-Formylchromone (Chromone- |
| | 3-carboxaldehyde) |
| 6-Chloro-3-formyl-7-methylchromone | 3-Formyl-6-isopropylchromone |
| 6-Chloro-7-methylchromanone | 3-Formyl-6-methylchromone |
| 6-Chloro-7-methylchromone | 6-Methoxy-3-formylchromone |
| 6-Chloro-7-methylchromone-2- | 6-Methylchromanone |
| carboxylic acid | |
| | 6-Methychromone |
| | 6-Methylchromone-2-carboxylic |
| | acid |

The term “mast cell stabilizer” as used herein is meant to encompass any compound with the ability to prevent or reduce mast cell activation and/or degranulation.

The term “inhibitor of mast cell-mediated inflammation” as used herein refers to any compound with the ability to prevent or mitigate the cascade of mast cell mediated events in an immunological response. Such compounds include, but are not limited to, mast cell stabilizers, inhibitors of platelet activating factor (PAF), inhibitors of eosinophil chemotactic factor (ECF), inhibitors of neutrophil chemotactic factor (NCF), and inhibitors of inflammatory cell recruitment, e.g., inhibitors of IL-4, IL-5, IL-16 or TNF-α. Inhibitors of mast cell mediated inflammation may also include inhibitors of a mast cell-mediated histamine cascade, including but not limited to inhibitors of histamine activity, inhibitors of leukotrienes (LTC₄), and inhibitors of prostaglandin D₂ (PGD₂).

The term “immune modulator” refers to any substance meant to alter the working of the humoral or cellular immune system of a subject. Such immune modulators include inhibitors of mast cell-mediated inflammation, interferons, interleukins, prostaglandins, steroids, cortico-steroids, colony-stimulating factors, chemotactic factors etc.

The term “sustained release” means release (of a drug) over an extended period of time, as contrasted with “bolus” release. Sustained release, for example, may be for a period of at least 12 hours, at least 24 hours, at least two weeks, at least a month, at least three months, or longer.

The term “drug delivery device” refers to any means for containing and releasing a drug wherein the drug is released into a subject. The means for containment is not limited to containment in a walled vessel, but may be any type of containment device, including non-injectable devices (pumps etc) and injectable devices, including a gel, a viscous or semi-solid material or even a liquid. Drug delivery devices are split into five major groups: inhaled, oral, transdermal, parenteral and suppository. Inhaled devices include gaseous, misting, emulsifying and nebulizing bronchial (including nasal) inhalers; oral includes mostly pills; whereas transdermal includes mostly patches. Parenteral includes two sub-groups: injectable and non-injectable devices. Non-injectable devices are generally referred to as “implants” or “non-injectable implants” and include e.g., pumps and solid biodegradable polymers. Injectable devices are split into bolus injections, that are injected and dissipate, releasing a drug all at once, and depots, that remain discrete at the site of injection, releasing drug over time. Depots include e.g., oils, gels, liquid polymers and non-polymers, and microspheres. Many drug delivery devices are described in Encyclopedia of Controlled Drug Delivery (1999), Edith Mathiowitz (Ed.), John Wiley & Sons, Inc.

The term “drug” as used herein, refers to any substance meant to alter animal physiology.

The term “dosage form” refers to a drug plus a drug delivery device.

The term “formulation” (or “drug formulation”) means any drug together with a pharmaceutically acceptable excipient or carrier such as a solvent such as water, phosphate buffered saline or other acceptable substance. A formulation may contain a drug such as an inhibitor of mast cell-mediated inflammation and other active agents, e.g., a compound that prevents eosiniphil recruitment, corticosteroids, a pain reliever etc. It may also contain an excipient, solvent or buffer or stabilizing agent.

The term “systemic delivery” is meant to encompass delivery (of a drug) to the systemic blood or lymph circulation of a subject and may encompass delivery by any parenteral route of delivery which permit drug to enter into the systemic circulation, e.g., intravenous, intra-arterial, intraperitoneal, intramuscular, subcutaneous, intra-adipose tissue, intra-lymphatic, etc.

The term “implanted” is used to mean placed within the body of a subject, e.g. placement within tissues or under the skin of a subject. It may be used to refer to things implanted by injection (depots) as well as to things implanted by other surgical means (non-injectable implants such as pumps).

The term “implantation site” means a site within the body of a subject at which a dosage form is placed.

The term “target site” means the intended site of action of a drug.

The term “non-injectable implant” means any non-injectable drug delivery device such as a pump, or a solid biodegradable or non-biodegradable polymer, such as an extruded polymer rod, a coaxial rod etc.

The term “depot” refers to any number of injectable controlled release drug-delivery systems. Depots include polymeric and non-polymeric materials, microsphere preparations, oils, emulsions and gels, and may be solid, liquid or semi-solid in form. For example, a depot as used in the present invention may be a high viscosity liquid, such as a non-polymeric non-water-soluble liquid carrier material, e.g., Sucrose Acetate Isobutyrate (SAIB) or another compound described in U.S. Pat. Nos. 5,747,058 and 5,968,542. For reference, please refer generally to Encyclopedia of Controlled Drug Delivery 1999, published by John Wiley & Sons Inc, edited by Edith Mathiowitz.

“Patterned” or “temporal” refers to delivery of drug in a pattern (in contrast with a bolus injection) e.g., at an increasing, decreasing, substantially constant, or pulsatile, rate or range of rates.

The terms “treat,” “treatment” and the like as used herein include prophylactic treatment and includes: (a) preventing inflammation from occurring in a subject that may be predisposed but is not at the time displaying symptoms; (b) inhibiting inflammation, e.g., arresting development of an inflammation-associated disease; or (c) relieving disease, i.e., causing regression and/or amelioration of the disease.

The phrase “to measurably inhibit inflammation” means to prophylactically prevent inflammation from occurring, such that, without the prophylaxis, the amount of inflammation would be greater than with such prophylaxis, and wherein the difference in inflammation is measurable to a reasonable scientifically measurable degree using standard methods, such as cytological assays (cell counts), chemical assays (e.g.: measuring the amount of histamine or other inflammatory factors in the blood), and clinical assays (such as pulmonary function testing (PFT) and forced expiratory flow volume (FEV). It is important to note that the “measurement of inflammation” as used in this disclosure includes measurement of early asthmatic response (EAR) and late asthmatic response (LAR), and of secondary indications of inflammation such as PFT and FEV. Measurable inhibition of inflammation in the present disclosure also includes reduction of pre-existing inflammation such that, without the treatment, the amount of inflammation would be greater than with the treatment, and wherein the difference is measurable to a reasonable scientifically measurable degree using standard methods.

Conditions Suitable For Treatment Using The Invention

Sustained-release administration of a drug according to the invention can be used to manage inflammation associated with any of a wide variety of disorders, including asthma, particularly disorders that require long-term therapy (treatment over a period of at least 12 hours, or days weeks or months). The invention may be used in a prophylactic manner. Inflammation amenable to therapy according to the invention may involve prolonged episodes alternating with relatively symptom-free intervals, or substantially unremitting inflammation that varies in severity.

Specific examples of conditions include, but are not necessarily limited to: autoimmune disorders, e.g., bursitis, rheumatoid arthritis, osteoarthritis, juvenile arthritis and lupus erythematosus; allergic ocular disorders, e.g., vernal kerato conjunctivitis, vernal conjunctivitis, and vernal keratitis; mastocytosis (cutaneous and systemic); nasal inflammation, e.g., allergic rhinitis and other allergic responses; gastrointestinal inflammation, e.g. inflammatory bowel disease such as ulcerative colitis and Crohn's disease; inflammatory gallbladder disease; pancreatitis; and respiratory inflammation e.g., adult respiratory distress syndrome (ARDS), pneumonia, bronchitis, cystic fibrosis, emphysema, viral infections, and various forms of asthma.

In one particular embodiment, the inflammation-associated disorder treated using the methods of the invention is asthma. Conventional asthma assessment and management are discussed in Pierson and Kacmarek's Foundations of Respiratory Care, pp. 215-217 and pp. 679-682. Asthma includes pediatric asthma, allergic asthma and exercise-induced asthma, and each of these may be present in the classified severities. The different severities of asthma are categorized as follows:

| Classification of | | | |
|---|---|---|---|
| Asthma Severity | Symptoms | Clinical Symptoms | Pulmonary Function |
| STEP 4 | Severe persistent | Continual symptoms | FEV1/PEFR is no greater |
| | | Limited physical activity | than 60% of predicted |
| | | Frequent exacerbations | PEFR variability > 30% |
| STEP 3 | Moderate | Daily symptoms | FEV1/PEFR exceeds 60% but |
| | Persistent | Daily use of inhaled short-acting β2-agonist | is less than 80% of predicted |
| | | Exacerbations affect activity. Exacerbations at least | PEFR variability exceeds |
| | | twice weekly and may last for days | 30% |
| STEP 2 | Mild persistent | Symptoms more frequent than twice weekly but less | FEV1/PEFR is at least 80% |
| | | than once a day. Exacerbations may affect activity | of predicted PEFR variability |
| | | | is between 20% and 30% |
| STEP 1 | Mild intermittent | Symptoms no more frequent than twice weekly | FEV1/PEFR is at least 80% |
| | | Asymptomatic and with normal PEFR between | of predicted |
| | | exacerbations. Exacerbations brief (hours to days) | PEFR variability is less than |
| | | Intensity of exacerbations varies. | 20% |

From “Guidelines for the Diagnosis and Management of Asthma.” Bethesda, Md: National Asthma Education and Prevention Program; 1997. National Institutes of Health publication 97-4051.

The present classification system is based on the described symptoms of asthma that are clinical features before treatment. The presence of one of the features of severity is sufficient to place a patient in that category, and an individual should be assigned to the most severe category in which any feature occurs. The characteristics noted in this table are general and may overlap because asthma is highly variable. Furthermore, an individual's classification may change over time. Patients at any level of severity can have mild, moderate, or severe exacerbations. Some patients with asthma experience severe and life-threatening exacerbations separated by long periods of normal pulmonary function and no symptoms.

Although inhibitors of mast cell-mediated inflammation such as sodium cromoglycate and nedocromil sodium are conventionally used to treat moderate persistent to mild intermittent asthma, the increased bioavailability of these agents using the methods and devices of the invention may also allow treatment of subjects with severe persistent asthma due to reduced administration problems and fewer bioavailability concerns than conventional inhalation therapy. For example, sodium cromoglycate administered using the devices and methods of the invention is almost completely bioavailable, compared with 7.5% bioavailability of inhaled sodium cromoglycate or less than 1% bioavailability of oral sodium cromoglycate. For all types of asthma, therapeutic intervention may occasionally or chronically require the use of the concurrent therapy, such as use of corticosteroids, in conjunction with the delivery of the inhibitor of mast cell-mediated inflammation.

Formulations Generally

A formulation is a drug plus a pharmaceutically acceptable excipient or carrier.

The present invention requires that a drug be formulated in certain ways in order to facilitate delivery by various exemplary methods described herein. Different formulations are required for pumps, polymer non-injectable implants, microspheres and SAIB depots.

A formulation of the invention generally comprises an inhibitor of mast cell-mediated inflammation, such as a chromone, and may additionally include a flavonoid (kaempferol), compounds that stimulate the phosphorylation of the 78 kD mast cell moesin, a piperazine (e.g., hydroxyzine), sodium cromoglycate, nedocromil sodium, disodium cromoglycate, a pyranoquinolone doxoantrazole, quercetin, tranilast, ketotifen, tiacrilast, azelastine, lodoxamide, mepyramine, and picumast.

Sodium cromoglycate, as well as techniques for the synthesis and various therapeutic applications thereof, is described, for example, in U.S. Pat. Nos. 4,161,516; 4,269,835; 4,882,170; 5,485,827; 5,509,404 and 5,766,633 (expressly incorporated by reference). For a review of the pharmacokinetics of sodium cromoglycate see, e.g., Saah M et al. (1996) J. Pharm Sci. 85:496-504, and Kato Y et al. (1999) Ann Allergy Asthma Immunol. 83:553-8. Nedocromil sodium, as well as technique for the synthesis and various therapeutic applications thereof, is described, for example, in U.S. Pat. Nos. 3,957,965; 4,161,516; 4,356,181; 4,590,206; 4,760,072; 4,866,072; 4,918,078; 4,935,244; 5,198,221; 5,248,493; 5,260,306 and 5,356,631 (expressly incorporated by reference).

The use of different inhibitors of mast cell-mediated inflammation suitable for the treatment of inflammation is reviewed in Melmon and Morrelli's Clinical Pharmacology: Basic Principles in Therapeutics, Problem-Based Therapeutic Decisions, pp. 250-253 (4^{th} Ed. 2000).

Inhibitors of mast cell-mediated inflammation can be provided by a variety of drugs, provided that such drug is substantially stable (i.e., not subject to degradation to an unacceptable amount at body temperature) for the pre-selected period of treatment. The concentration of an inhibitor of mast cell-mediated inflammation in the formulation may vary from about 0.1 wt. % to about 50 or 75 wt. %. The drug can be provided in any form suitable for sustained delivery, e.g., solid, semi-solid, gel, liquid, suspension, emulsion, osmotic dosage formulation, diffusion dosage formulation, erodible formulation, depot injectable form, etc. Of particular interest is the administration of an inhibitor of mast cell mediated inflammation from a depot or an osmotic pump or from an elemental osmotic pump.

Organic or inorganic carriers, excipients and/or diluents suitable for parenteral use can be included in the formulations. Such physiologically acceptable carriers are well known in the art. Exemplary liquid carriers can be sterile non-aqueous or aqueous solutions which contain no materials other than the active agent or agents. The formulations can optionally further comprise buffers such as sodium phosphate at physiological pH, physiological saline or both (i.e., phosphate-buffered saline). Suitable aqueous carriers may optionally further comprise more than one buffer salt, as well as other salts (such as sodium and potassium chlorides) and/or other solutes.

Suitable excipients can comprise dextrose, glycerol, alcohol (e.g., ethanol), and the like, and combinations of one or more thereof with vegetable oils, propylene glycol, polyethylene glycol, benzyl alcohol, benzyl benzoate, dimethyl sulfoxide (DMSO), organics, and the like to provide a suitable composition. In addition, if desired, the composition can comprise nonionic or ionic surfactants, dispersing agents, wetting or emulsifying agents, isotonic agents, pH buffering agents, and dissolution promoting agents, stabilizers, antiseptic agents and other typical auxiliary additives employed in the formulation of pharmaceutical preparations. Exemplary additional active ingredients include, but are not limited to, an analgesic, an opioid, glucocorticoids, long-acting beta2-agonists, nonsteroidal anti-inflammatory drugs, methylxanthines, leukotriene receptor modifiers, phosphodiesterase inhibitors, antitussives, an antimicrobial agent, an antimucosal agent, an antiviral agent, and/or an antioncogenic agent. The biologically active agent may optionally be esterified or complexed with polyethelyne glycol (“PEGylated”) to provide enhanced bioavailability or sustained release characteristics. The biologically active agent may optionally be mixed with a biodegradable polymer prior to mixing with the other excipient or excipients. This mixing may be accomplished by solvent or melt blending and then grinding to the proper particle size.

Sodium cromoglycate may be used in the form of a free acid, sodium or other monovalent salt, zinc, calcium or other divalent salt, or as a pro-drug such as an ester or amide form. Sodium cromoglycate may be obtained commercially from Hawkins Pharmaceuticals.

Implantation Of The Drug Delivery Device

A depot or non-injectable implant containing the drug formulation can be introduced into a subject at any suitable site using methods and devices well known in the art. Implantation sites include, but are not necessarily limited to a subdermal, subcutaneous, intramuscular, or other suitable site within a subject's body, for example, subcutaneously in the upper arm. Subcutaneous implantation sites are preferred because of convenience. In some embodiments, the implantation site is at or near the target site (the site at which the drug acts). In some embodiments, the target site is distant form the implantation site. Delivery of drug from a drug delivery device at an implantation site that is distant from a target site can be accomplished by providing the drug delivery device with a catheter, as described in more detail below.

Target sites compatible with systemic delivery include, but are not necessarily limited to, subcutaneous, intravenous, intra-arterial, intra-muscular, intra-adipose tissue, intra-lymphatic and sublingual sites. Exemplary subcutaneous target sites include external subcutaneous sites (e.g., under the skin of the arm, shoulder, neck, back, or leg) and internal subcutaneous sites within a body cavity (e.g., within the mouth). In addition, the target site can be the desired site of action for the inflammation or the locus of the inflammation (e.g., lung tissue, bowels, joints, etc).

An example of implantation for a SAIB depot form would be to inject a depot subcutaneously into the upper are of a subject using a needle and a standard syringe. Once the needle is withdrawn, the depot remains under the skin and becomes more viscous and gelatinous as hydrophilic solvent is released from the bulk of the hydrophobic matrix into surrounding tissue. From this stable location, the depot then releases sodium cromoglycate at a relatively steady rate into the surrounding tissue, from where the drug finds its way into the circulatory system, and thence to its site of action, on the surface of mast cells throughout the circulation and in the bronchioles. The depot may release the drug for many weeks or months, and will eventually be degraded by the natural mechanisms of the body, leaving no trace of the drug delivery device.

An example of implantation of a pump would be to surgically implant a DUROS® pump subcutaneously in the upper arm of a subject by making a small incision and using a trocar, under local anesthetic.

Duration And Rate Of Delivery Of Inhibitors Of Mast Cell-Mediated Inflammation

The drug formulation is delivered at a dose that is therapeutically effective to reduce inflammation (e.g., sufficient to accomplish substantial prophylaxis of symptoms). In general, administration of inhibitor of mast cell-mediated inflammation according to the invention continues for several hours, for example at least 12 hours, at least 24 hours, at least a week, at least two weeks, at least a month, at least three months, six months or longer. A formulation may be delivered to the subject for a pre-selected period without the need for introducing a new dosage form or replenishing the formulation contained in the dosage form.

In general, suitable drug delivery devices are those that can deliver drug at a low dose/delivery rate, e.g., for sodium cromoglycate or nedocromil sodium, from about 0.1 μg/hr to about 200 μg/hr, and in some instances up to about 833 μg per hour, from 0.01 mg/day to 20 mg/day, and preferably at a low volume rate e.g., on the order of nanoliters to microliters per day. In one embodiment, a volume rate of from about 0.01 μl/day to about 2 ml/day is accomplished by delivery of about 80 μl/hour over a period of 24 hours, with the delivery rate over that 24 hours period fluctuating over that period by about ±5% to 10%. Preferred osmotically-driven drug release systems can provide for release of drug in a range of rates of from about 0.1 μg/hr to about 200 μg/hr, and which can be delivered at a volume rate of from about 0.25 μl/day to about 100 μl/day, preferably from about 0.04 μl/day to about 10 μl/day, generally from about 0.2 μl/day to about 5 μl/day, typically from about 0.5 μl/day to about 1 μl/day. Generally, the delivery rate is substantially constant (± about 5% to 10% of the cited volume over the cited time period). Delivery of a formulation may be in a patterned or substantially continuous fashion.

Drug Delivery Devices And Dosage Forms

A drug delivery device includes any means for containing and releasing a drug wherein the drug is released into a subject. A dosage form is a drug plus a drug delivery device. Drug delivery devices include injectable devices (depots).

A variety of parenteral sustained-release drug delivery devices can be used in the present invention to accomplish delivery of a drug. Exemplary drug delivery devices include injectable devices (depots e.g., non-polymers, gels, oils, microspheres, liposomes, hydrogels etc), and implanted devices (e.g., pumps and erodible polymer rods). See the Encyclopedia of Controlled Drug Delivery (1999), Edith Mathiowitz (Ed.), John Wiley & Sons, Inc.

The invention contemplates sustained-release of a drug. The drug is generally released systemically from an implanted device into the subject to treat inflammation, although the drug may also be delivered directly to the site of action e.g., joint, lungs etc via a catheter. Generally, an inhibitor of mast cell-mediated inflammation is administered to an individual for at least 12 hours to at least a week. In certain embodiments, the dosage forms of the invention may be designed to deliver a drug for at least 10, 20, 30, 100 days or at least 4 weeks, or at least 3 months, or at least 6 months or more, as needed. Sustained release may be accomplished by incorporation of drug into a polymer or non-polymer or microsphere depot, incorporation of drug in a biodegradable polymer, using an osmotically-driven device, etc. Other well known methods employ pumps such vapor pressure pumps, electrolytic pumps, effervescent pumps, piezoelectric pumps, electrochemical pumps (such as corrosion-type pumps), and osmotic pumps.

In general, the drug delivery device must be capable of carrying the drug formulation in such quantities as therapeutically required for treatment over the pre-selected period, and must provide sufficient protection to the formulation from degradation by body processes for the duration of treatment. For example, the drug delivery device may be a pump, with an exterior of a protective solid material resistant to leakage, cracking, breakage, or distortion to prevent expelling of the drug in an uncontrolled manner. Suitable materials for making the components of such a device are well known in the art, for example one may use a non-reactive polymer or a biocompatible metal or alloy. Suitable polymers include, but are not necessarily limited to, acrylonitrile polymers such as acrylonitrile-butadiene-styrene polymer, and the like; halogenated polymers such as polytetrafluoroethylene, polyurethane, polychlorotrifluoroethylene, copolymer tetrafluoroethylene and hexafluoropropylene; polyethylene vinylacetate (EVA), polyimide; polysulfone; polycarbonate; polyethylene; polypropylene; polyvinylchloride-acrylic copolymer; polycarbonate-acrylonitrile-butadiene-styrene; polystyrene; cellulosic polymers; and the like. Further exemplary polymers are described in The Handbook of Common Polymers, Scott and Roff, CRC Press, Cleveland Rubber Co., Cleveland, Ohio.

Metallic materials suitable for making a pump include stainless steel, titanium, platinum, tantalum, gold and their alloys; gold-plated ferrous alloys; platinum-plated titanium, stainless steel, tantalum, gold and their alloys as well as other ferrous alloys; cobalt-chromium alloys; and titanium nitride-coated stainless steel, titanium, platinum, tantalum, gold, and their alloys.

A pump used with the invention may be osmotic pump, an electroosmotic pump, a vapor pressure pump, an electrolytic pump, an effervescent pump, a piezoelectric pump etc. Drug delivery devices based upon a mechanical or electromechanical infusion pump, can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Osmotic pumps are sometimes preferred due to their combined advantages of more consistent controlled release and relatively small size. Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423, 5,112,614; 5,137,727; 5,234,692; 5,234,693; 5,728,396; 5,985,305; and the like.

In certain embodiments employing a pump, the drug delivery device may include a catheter. In such a case the drug is delivered through the catheter to the target site as a result of pressure generated within the device, or by capillary action, or by diffusion, by electrodiffusion or by electroosmosis through the device and/or the catheter.

Polymeric matrices may also be used as drug delivery devices, and may be made of polymers, including biostable polymers and biodegradable polymers, Such polymers may be liquid or solid and may be formed into monolithic or coaxially extruded rods impregnated with drug. Exemplary biostable polymers include silicone, polyurethane, polyether urethane, polyether urethane urea, polyamide, polyacetal, polyester, poly ethylene-chlorotrifluoroethylene, polytetrafluoroethylene (PTFE or “Teflon™”), styrene butadiene rubber, polyethylene, polypropylene, polyphenylene oxide-polystyrene, poly-a-chloro-p-xylene, polymethylpentene, polysulfone and other related biostable polymers. Exemplary biodegradable polymers include, but are not necessarily limited to, polyanhydrides, cyclodestrans, polylactic-glycolic acid, polyorthoesters, n-vinyl alcohol, polyethylene oxide/polyethylene terephthalate, polyglycolic acid, polylactic acid and other related bioabsorbable polymers.

Non-polymer matrices may be made from SAIB or similar materials, with suitable solvents, as discussed herein.

In certain embodiments, the drug delivery device may be a biodegradable monolithic rod. An experimental example of one such embodiment is a monolithic rod prepared by melt extrusion of a sodium cromoglycate-polymer mixture using, as the polymer poly (dl-lactide-co-glycolide) or poly (caprolactone). Other polymers that may be used are listed under the discussion of coaxial rods. The extruded rod is implanted in the subject using standard surgical techniques under local anesthetic.

In certain embodiments, the drug delivery device may be a coaxial rod, in which there is drug in the core as well as the sheath. In this disclosure, the inventors disclose, as an example, a specific coaxial rod, which is believed to be novel. Note that the disclosed coaxial rods may be used to deliver any manner of drug, and sodium cromoglycate is used here merely as an example fitting and convenient to the present disclosure. It is believed that the method for making the disclosed coaxial rod, as well as the rod itself, is novel. The polymer used could be any suitable polymer, which would be easily determinable by one of skill in the art, for example polyhydroxy acids, such as poly(lactide)s, poly(glycolide)s, poly(lactide-co-glycolide)s, poly(lactic acid)s, poly(glycolic acid)s, and poly(lactic acid-co-glycolic acid)s, polyanhydrides, polyorthoesters, polyetheresters, polycaprolactone, polyesteramides, polyphosphazines, polycarbonates, polyamides, and copolymers and blends thereof. A preferred material is polycaprolactone, and is used in the example disclosed herein. The extruded rod is implanted in the subject using standard surgical techniques under local anesthetic.

In certain embodiments, the drug delivery device/dosage form is an injectable depot. A depot encompasses any number of injectable materials that may act as a reservoir for a drug, and from which a drug is released in a controlled manner. A depot may include microparticles, microspheres, liposomes, polymeric and non-polymeric biodegradable materials that may be high viscosity liquids, or may be solid when implanted. A depot is generally placed subcutaneously by injection. Depots are generally thickish liquids prior to injection (using a large gauge needle), but may become more viscous, or even semi-solid after injection, as solvents are released leaving a thick hydrophobic matrix.

### EXAMPLES

Delivery Of Drug Using An Implanted Pump Without A Catheter

Sodium cromoglycate may be delivered from an implanted pump without the use of a catheter. For example, a DUROS® osmotic pump could be used. Sodium cromoglycate (Hawkins Pharmaceuticals) may be formulated by dissolving the sodium cromoglycate in sterile distilled water to a maximum solubility of about 120 mg/ml, and adjusting pH to about pH 7 sodium hydroxide. The formulation of sodium cromoglycate may then be loaded into a DUROS® pump under aseptic conditions. The pump is implanted in a subject, for instance subcutaneously in the upper arm, under local anesthetic and the drug would be released therefrom into the body of the subject.

[image]

Delivery Of Drug Using An Implanted Pump And Catheter

In some embodiments it may be desirable to provide a drug delivery catheter with the drug delivery device, e.g., where the implantation site and the desired target site are different. The drug delivery catheter generally has a first end (or “proximal” end) associated with the drug delivery device, and a second end (or “distal” end) for delivery of the drug to a desired target site. The formulation contained in a drug reservoir can move into the drug delivery catheter, and out a delivery outlet of the catheter which is positioned at the desired target site. Where the drug delivery device dispenses drug by convection, the size of the catheter lumen leading from the reservoir of the drug release system can be designed as described by Theeuwes (1975) J. Pharm. Sci. 64:1987-91. The distal end of the drug delivery catheter can provide a distinct opening for delivery of drug, or as a series of openings.

The drug delivery catheter may be produced from any of a variety of suitable materials, e.g. (but not limited to) polymers; metals; glasses; polyolefins (high density polyethylene (HDPE), low density polyethylene (LDPE), linear low density polyethylene (LLDPE), polypropylene (PP), and the like); nylons; polyethylene terephtholate; silicones; urethanes; liquid crystal polymers; PEBAX®; HYTREL®; TEFLON®; perflouroethylene (PFE) perflouroalkoxy resins (PFA); poly(methyl methacrylate) (PMMA); multilaminates of polymer, metals, and/or glass; nitinol; and the like.

In one embodiment, the drug delivery catheter is primed with a drug-comprising formulation, e.g., is substantially pre-filled with drug prior to implantation. Priming of the drug delivery catheter reduces delivery start-up time, i.e., time related to movement of the drug from the drug delivery device to the distal end of the drug delivery catheter. This feature is particularly advantageous when low flow rates are desired.

The drug delivery device to which the catheter may be attached maybe any of those listed in this disclosure including an osmotic pump such as the DUROS® pump.

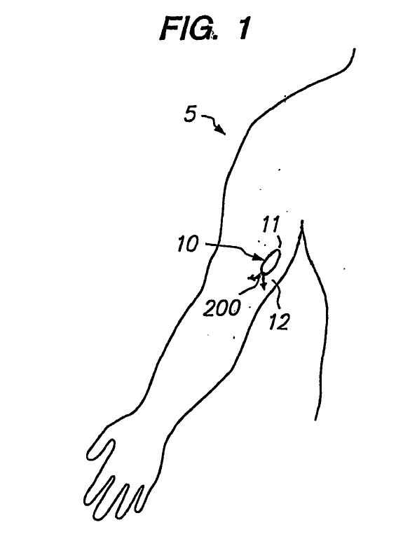

FIG. 1 illustrates one embodiment of the invention, wherein a formulation is delivered from an implanted drug delivery device that provides for sustained release of a formulation from a drug reservoir to a subcutaneous site. In this example, the drug delivery device 10 is implanted at a subcutaneous site in the patient's arm 5. Arrows 200 illustrates flow of drug from the device's drug reservoir to the subcutaneous sites.

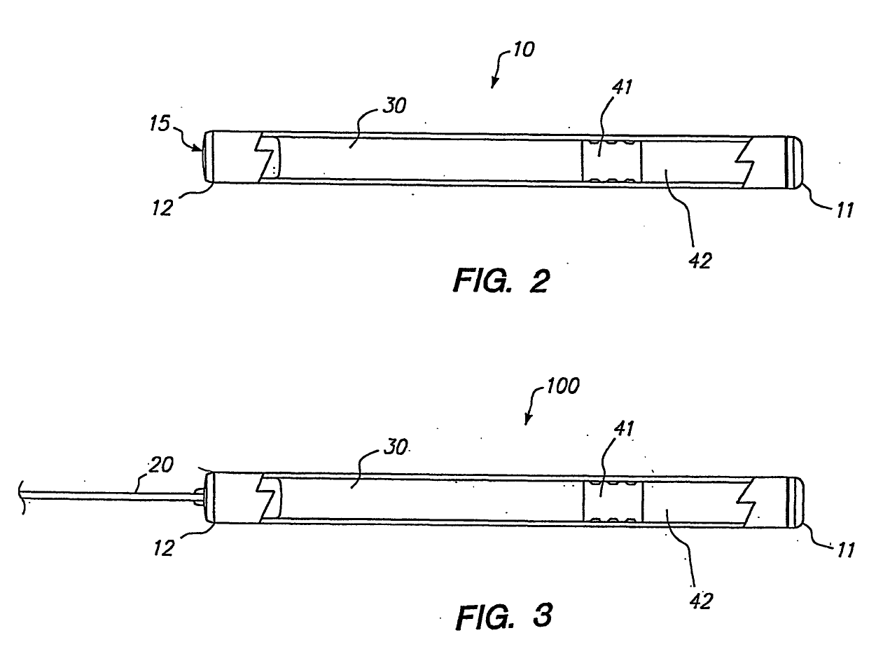

FIG. 2 provides a view of a pump 10. The pump comprises proximal and distal ends, 11 and 12, with the distal end defining an orifice 15 through which drug exits from the drug reservoir 30 for delivery to the subcutaneous site. Pressure is provided by an osmotic engine comprising a piston 41 and a chamber comprising an osmotic engine 42.

FIG. 3 shows a pump 100 with catheter 20. The walls of the drug delivery catheter define a lumen, and the drug delivery catheter is associated with the pump 100 so that a drug delivery pathway is provided from the drug reservoir 30, through the distal end 12 and out through the catheter 20. The catheter can be positioned for systemic delivery of drug, for example, subcutaneously. Or for delivery of a drug to a specific site.

Methods for implanting or otherwise positioning the dosage forms of the invention into the body are well known in the art and are generally performed under aseptic conditions with local or general anesthesia. Removal and/or replacement of the dosage forms, if necessary, can be accomplished likewise.

Delivery Of Drug Using An SAIB Depot

Sodium cromoglycate may be delivered from a SAIB depot injected subcutaneously under the skin of the upper arm of a subject. SAIB may be mixed with one or more suitable solvents which may be hydroxylic or nonhydroxylic and which may be used alone or in combination. Examples of solvents include ethanol, NMP, benzyl benzoate, benzoic acid, ethyl lactate, proplyene carbonate, glycofurol and Miglyol 810, or mixtures thereof. The solvent can be added to SAIB in a ratio of from about 5%-65% by weight solvent, usually less than 50% by weight. The active agent, for example sodium cromoglycate in a lypholized or dry powder form, may then be added to the SAIB/solvent mixture. The mixture is then mixed until homogeneous. The resulting depot is then ready for injection into the subject.

Experimental formulation examples of SAIB depots include the following:

In one example, 0.5 g sodium cromoglycate (Hawkins Pharmaceuticals) was mixed with 9.5 g of a 70:30 mixture of SAIB (Eastman Chemical Co.): glycofurol until a homogeneous mixture is achieved. Accurately weighed samples of the formulation were injected into 250 mL of dissolution buffer (BS, 0.01M, pH 7.4 with sodium azide) prewarmed to 37° C. in a 250-mL round bottom flask. The flasks were agitated at 125 rpm in an orbital shaker. Samples (3 mL) were removed at 0.25, 0.5, 1, 2, 3, 4, 5, 6 hr, 24 hr and daily thereafter. The samples were assayed for sodium cromoglycate using UV-visible spectrometry at 326 nm. This depot resulted in 100% release of drug in a 24 hr period.

Another depot formulation was prepared by first preparing 9.17 g of a 50:50 mixture of SAIB and Miglyol 810. To this mixture was added 0.83 g of a mixture of sodium cromoglycate and polycaprolactone (PCL) (Birmingham Polymers, Inc.) in a ratio of 3 parts cromolyn to 2 parts PCL. The sodium cromoglycate and PCL mixture was prepared by dissolving 12 grams of PCL in 100 mL of methylene chloride and adding 18 grams of cromolyn to the solution. The solution was poured onto aluminum foil and the methylene chloride evaporated. The resulting film was ground into particles and the particles were added to the SAIB/benzyl benzoate mixture. The resulting depot formulation was a suspension. Samples of this depot formulation were incubated and assayed to determine the rate of sodium cromoglycate. This formulation released a cumulative total of about 80% of the available sodium cromoglycate over a 10-day period. A similar formulation with 30% sodium cromoglycate gives a longer period of release.

As an additional example, the calcium salt of sodium cromoglycate is prepared by adding sodium cromoglycate solution to a 6.35 m mole/mL CaCl₂ solution. The calcium salt is added to a solution of poly(lactic acid) (Birmingham Polymers, Inc.) in methylene chloride. The methylene chloride is evaporated and the resulting film is ground to form particles which are then added to a mixture of SAIB:benzyl benzoate. The final formulation is 45:45:10 SAIB:benzyl benzoate:poly(lactic acid) with 5% wt sodium cromoglycate. This formulation releases 100% sodium cromoglycate in vitro over a 30-day period, assayed as described above.

In a further example, a formulation of the free acid of sodium cromoglycate (Kemprotec) is mixed with a solution of poly(lactic acid) in methylene chloride. After preparing particles of the free acid and poly(lactic acid) as described above, a 7.5% wt sodium cromoglycate free acid depot formulation in SAIB:benzyl benzoate:poly(lactic acid) (45:45:10) is prepared. This formulation releases sodium cromoglycate in vitro over a period of 60 days.

Having formulated a SAIB depot it may be injected subcutaneously into the upper arm of a subject using a needle and a standard syringe. Alternatively, other parenteral routes of administration may be used. The injection volume and needle size are chosen to optimally achieve the desired rate and duration of release of active agent while minimizing discomfort to the patient. Once the needle is withdrawn, the depot remains under the skin and becomes more viscous as solvent is released from the bulk of the hydrophobic matrix into surrounding tissue.

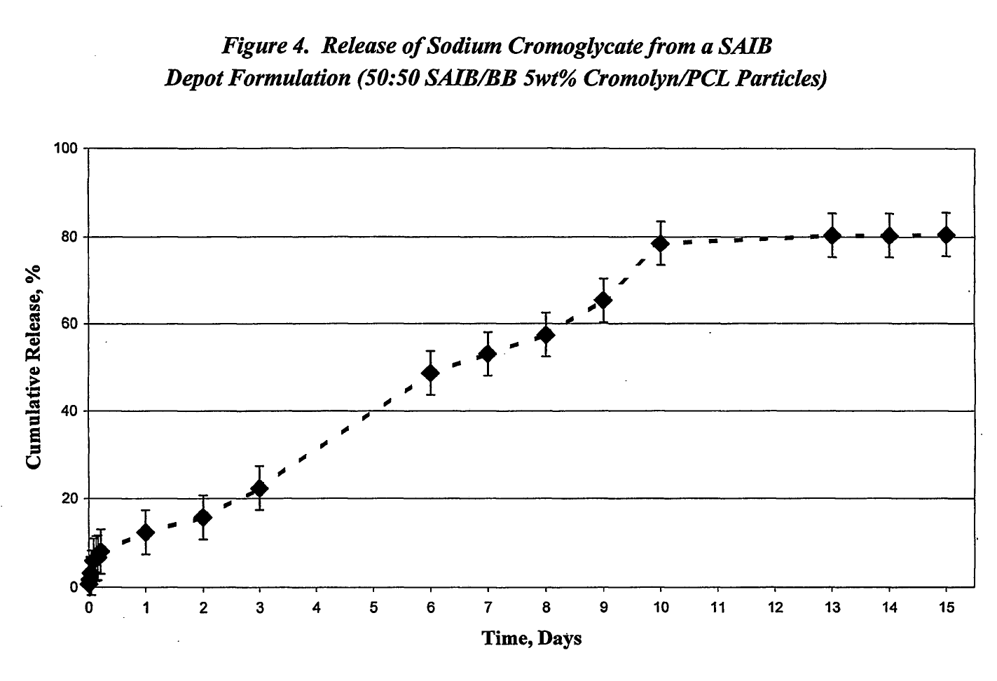

From this stable location, the depot then releases sodium cromoglycate at a relatively steady rate into the surrounding tissue (FIG. 4), from where the drug finds its way into the circulatory system, and thence to its site of action at mast cells throughout the circulation and in the bronchioles. The depot may release the drug for many weeks or months, and will eventually be degraded by the natural mechanisms of the body, leaving no trace of the drug delivery device.

Delivery Of Drug Using A Monolithic Rod

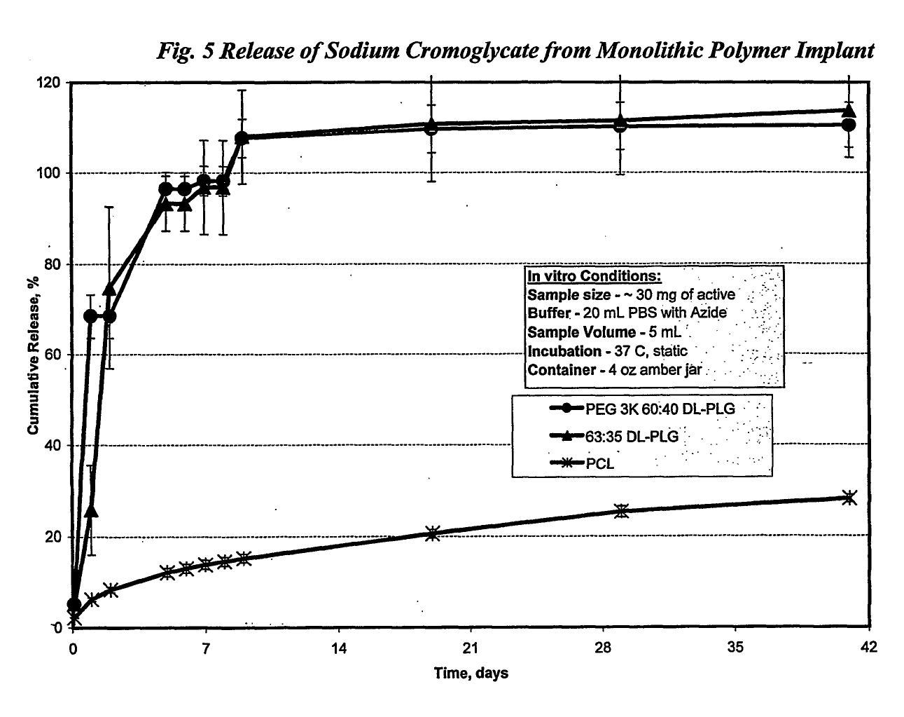

A biodegradable monolithic rod may also be used (FIG. 5). An experimental example of one such embodiment is a monolithic rod prepared by melt extrusion at 95° C. using a Tinius Olsen extruder, wherein the rod contained 30% by weight sodium cromoglycate within a polymer of either 65:35 poly (dl-lactide-co-glycolide) (65:35 lactide to glycolide ratio) or a PEG-containing 60:40 poly (dl-lactide-co-glycolide) (60:40 lactide:glycolide ratio). The extruded rods were assayed for release of sodium cromoglycate by placing in 40 mL of dissolution buffer (PBS, 0.01M, pH 7.4 with sodium azide) in a 120 or 240-mL amber bottle at 37° C., with no agitation. After incubation for 1 hr 5 mL of buffer is removed for analysis and replaced with fresh buffer. Samples are removed for analysis daily for one week and weekly thereafter. Analysis was by UV spectrometry at 326 nm. This rod resulted in 100% release over 7 days. A similarly prepared rod made from poly (caprolactone) showed a 14% release over 7 days and a 25% release over 29 days.

Delivery Of Drug Using A Coaxial Rod

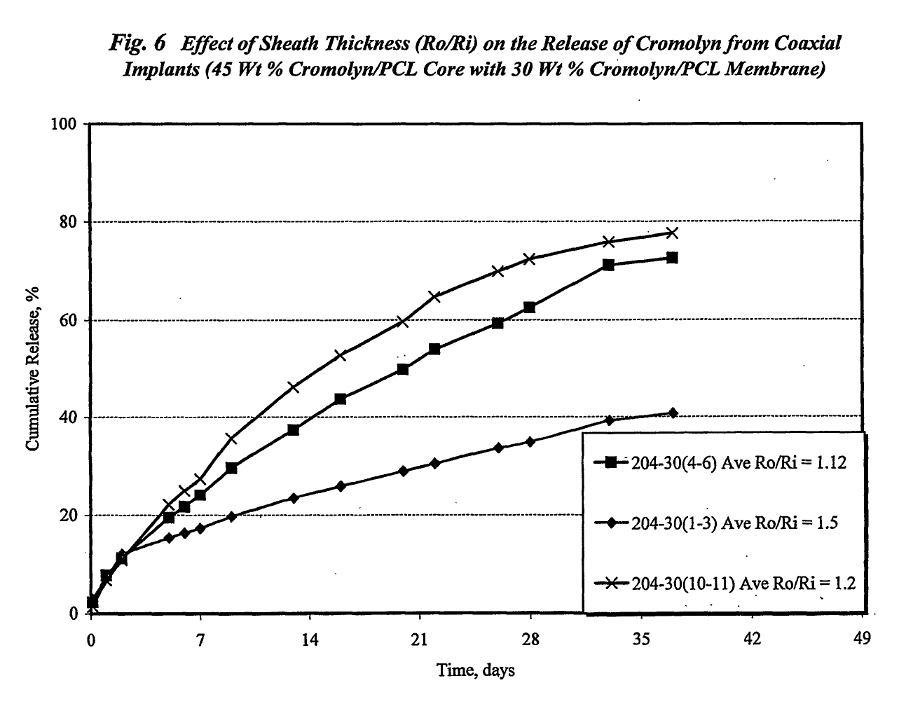

A novel coaxial rod may also be used (FIG. 6), in which there is drug in the core as well as the sheath. An experimental example of one such embodiment is a novel coaxial rod in which sodium cromoglycate and the core excipient (for example polycaprolactone, Birmingham Polymers, Inc.) were solvent-blended to ensure a uniform mixture of the drug and polymer.

The drug and excipient polymer were dissolved in dichloromethane, and the resulting solution was then cast as a thin film onto a sheet of Teflon. The film was dried under vacuum and then cryogenically ground using a Retsch Ultra Centrifugal Mill Model ZM 100 equipped with a 1.0-mm screen. The ground material was further dried under vacuum for at least 24 hrs. prior to extrusion.

Coaxial rods were prepared by operating two ⅜” Randcastle Microtruders simultaneously. The ground material was processed via the first extruder and then fed through the center orifice of the coaxial die to form the core of the implant. Pure polymer was fed through the second extruder to the outer, concentric ring of the coaxial die to form the rate-controlling membrane polycaprolactone, Birmingham Polymers, Inc.). The thickness of the membrane was manipulated by adjusting the speed of the second extruder relative to the speed of the first extruder.

The resulting extruded rods had core of 45% wt cromolyn in polycaprolactone with a membrane sheath of 30% wt cromolyn in polycaprolactone. The rods were cut to length (2 cm) and the outer radius (Ro) and inner radius (Ri) determined by measuring a cross section of the rod under a light microscope. The rods were assayed for sodium cromoglycate release by placing in 40 mL of dissolution buffer (PBS, 0.01 M, pH 7.4 with sodium azide) in an amber bottle at 37° C. with no stirring. After incubation for 1 hr, 5 mL of buffer was removed for analysis and replaced with fresh buffer. Samples were removed (with replacement by fresh buffer) for analysis daily for one week and weekly thereafter. Analysis for drug was by UV spectrometry at 326 nm.

The resulting rods released sodium cromoglycate over a 35-day period in amounts ranging from about 40% of the drug loading for rods with a Ro/Ri of 1.5%, to about 80% of the loading for rods with a Ro/Ri of 1.2 (FIG. 6).

1. A wholly implantable sustained-release dosage form for the inhibition of inflammation in a subject, said dosage form comprising a drug delivery device and an inhibitor of mast cell-mediated inflammation, wherein said inhibitor of mast cell-mediated inflammation is released from said drug delivery device, for a period of at least seven days, in an amount sufficient to measurably inhibit inflammation in said subject.

2. The dosage form of claim 1, wherein said drug delivery device is selected from the group consisting of: a non-injectable implant and a depot.

3. The dosage form of claim 1, wherein said drug delivery device is a depot.

4. The dosage form of claim 3, wherein said inhibitor of mast cell-mediated inflammation is a chromone.

5. The dosage form of claim 4, wherein said depot comprises sucrose acetate isobutyrate.

6. The dosage form of claim 1, wherein said drug delivery device is a pump.

7. An implantable sustained-release dosage form for the delivery of a drug to a subject, said dosage form comprising a coaxial rod and a drug, wherein said drug is continuously released from said coaxial rod for a period of at least seven days, and wherein said coaxial rod comprises a core surrounded by a sheath, and wherein both said sheath and said core comprise a polymer, and wherein both said sheath and said core further comprise said drug.

8. The dosage form of claim 7, wherein said polymer is selected from a group consisting of: polyhydroxy acids, such as poly(lactide)s, poly(glycolide)s, poly(lactide-co-glycolide)s, poly(lactic acid)s, poly(glycolic acid)s, and poly(lactic acid-co-glycolic acid)s, polyanhydrides, polyorthoesters, polyetheresters, polycaprolactone, polyesteramides, polyphosphazines, polycarbonates, polyamides, and copolymers and blends thereof.

9. The dosage form of claim 8, wherein said polymer comprises polycaprolactone.

10. The dosage form of claim 9, wherein said drug comprises a chromone.
